(19) United States
(12) Patent Application Publication
(10) Pub. No.: US 2011/0008867 A1
(43) Pub. Date: Jan. 13, 2011

(54) COMPOSITIONS AND METHODS FOR THE PRODUCTION OF A COMPOUND

(75) Inventors: Andrey J. Zarur, Winchester, MA (US); James Swartz, Menlo Park, CA (US)

Correspondence Address: EDWARDS ANGELL PALMER & DODGE LLP, P.O. BOX 55874, BOSTON, MA, 02205, US

(73) Assignee: GREENLIGHT BIOSCIENCES, Medford, MA (US)

(21) Appl. No.: 12/644,998

(22) Filed: Dec. 22, 2009

Related U.S. Application Data
(60) Provisional application No. 61/140,053, filed on Dec. 22, 2008.

(51) Int. Cl. C12N 9/12; C12N 9/00; C12N 9/14; C12N 9/88; C12N 9/02; C12N 9/90

(52) U.S. Cl. 435/189; 435/183; 435/195; 435/232; 435/194; 435/233

(57) ABSTRACT

The invention features compositions and methods that are useful for the production of a compound.

### CROSS-REFERENCE TO RELATED APPLICATION

This application claims the benefit of U.S. Provisional Application No. 61/140,053, filed on Dec. 22, 2008, the entire contents of which are incorporated herein by reference.

### BACKGROUND OF THE INVENTION

Despite progress in industrial fermentation and advances in biotechnology, present methods do not allow for the production of large scale (e.g., industrial or commercial scale) quantities of certain compounds. For example, most linear and aromatic hydrocarbons, e.g., generated in the production of biofuels, are strong inhibitors of cell growth. As a result, biochemical synthesis is limited to natural products (e.g., vitamins, enzymes, antibodies) and high-value, low-volume synthetics. Nevertheless, there is a large market for carbon-neutral synthetic products and cytotoxic products, which cannot be produced by industrial fermentation. Thus, there is a need for alternate production methods that provide for the large scale production of difficult to synthesize compounds, including therapeutically beneficial compounds such as biocides, antibiotics, and anticancer compounds.

The Centers for Disease Control and Prevention (CDC) estimate that between 5 percent and 20 percent of Americans will develop the flu each year. According to the CDC, about 200,000 people will be hospitalized due to flu complications, and as many as 36,000 will die as a result of the illness. Those most at risk from influenza are the elderly, children, and people with chronic health conditions. Avian influenza which normally infects birds, including poultry poses a greater and more specific concern. Because these viruses do not commonly infect humans, there is little or no immune protection against them in the human population. Following outbreaks of avian influenza, i.e., Influenza A (H5N1) virus, in poultry in Asia during late 2003 and early 2004, more than 130 human cases have been reported by the World Health Organization since January 2004, and 70 people have so far died as a result of infection with avian influenza.

Oseltamivir is a neuraminidase inhibitor used in the treatment and prophylaxis of both influenza A and influenza B, and is widely considered the most useful treatment against avian flu. Oseltamivir phosphate, the first orally active, commercially developed neuraminidase inhibitor, was developed by Gilead Sciences and is currently marketed by Hoffman-La Roche (Roche) under the trade name TAMIFLU. As an inhibitor of neuraminidase, which is essential for influenza virus replication, TAMIFLU is potent against the H5N1 and H7N7 virus strains. TAMIFLU was widely used during the H5N1 avian influenza epidemic in Southeast Asia in 2005. However the current supply of TAMIFLU is estimated to cover only 2% of the world population. The major bottleneck in TAMIFLU production is the availability of shikimic acid, a naturally occurring chemical compound derived from plants. Although chemical reactions for synthesizing shikimic acid are known, current methods to synthesize shikimic acid involve potentially explosive azide chemistry and it is difficult to synthesize shikimic acid in large quantities on a commercial scale.

National governments and the medical community worldwide have recognized the urgent need for the mass production and stockpiling of therapeutic agents, such as TAMIFLU, in response to possible global pandemics. For example, despite progress in therapies for influenza, present methods for treating influenza are inadequate to emergencies on a global or even national scale. Therefore, there is an urgent need for the production of compounds useful in the treatment of infectious diseases.

### SUMMARY OF THE INVENTION

As described below, the present invention features compositions and systems that provides for the production of a compound of interest, such as a therapeutically useful compound or precursor thereof.

In one aspect, the invention provides an isolated composition containing an enzyme that catalyzes the formation of a compound and an adenosine triphosphate (ATP) regeneration system, where the enzyme and the ATP regeneration system are obtained from the same source or separate sources, and where at least one is a cell-free extract.

In another aspect, the invention provides an in vitro cell-free system for the synthesis of a compound, the system containing a carbon source, a phosphate source, water, an enzyme that catalyzes the formation of a compound, an energy source, adenosine diphosphate (ADP), and an adenosine triphosphate (ATP) regeneration system.

In yet another aspect, the invention provides a method for synthesizing a compound, the method involving providing the components including a source of carbon, a source of phosphate, water, enzymes that catalyze the formation of a compound, an energy source, adenosine diphosphate (ADP), and an adenosine triphosphate (ATP) regeneration system to form an in vitro cell-free reaction; and incubating the in vitro cell-free reaction to synthesize the compound.

In an additional aspect, the invention provides a method for synthesis of shikimic acid, the method involving providing the components including a precursor for shikimic acid, a source of ATP, and enzymes that catalyze the formation of shikimic acid to form an in vitro cell-free reaction; and incubating the in vitro cell-free reaction, thereby synthesizing shikimic acid.

In another aspect, the invention provides an isolated composition comprising an enzyme selected from the group consisting of 3-deoxy-D-arabino-heptulosonate (DAHP) synthase, dehydroquinate synthase, dehydroquinate dehydratase, dehydroshikimate dehydrogenase, or combinations thereof, and an adenosine triphosphate ATP regeneration system, where the enzyme and the ATP regeneration system are obtained from the same source or separate sources, where at least one source is a cell-free extract.

In still another aspect, the invention provides an in vitro cell-free system for the synthesis of a compound containing a carbon source, a phosphate source, water, an energy source, adenosine diphosphate (ADP), a photosynthetic adenosine triphosphate (ATP) regeneration system, and an enzyme including 3-deoxy-D-arabino-heptulosonate (DAHP) synthase, dehydroquinate synthase, dehydroquinate dehydratase, dehydroshikimate dehydrogenase, or combinations thereof.

In yet another aspect, the invention provides a method for synthesizing a compound, the method involving providing the components including a source of carbon, a source of phosphate, water, adenosine diphosphate (ADP), an adenosine triphosphate (ATP) regeneration system, and an enzyme selected from the group consisting of 3-deoxy-D-arabino-heptulosonate (DAHP) synthase, dehydroquinate synthase, dehydroquinate dehydratase, dehydroshikimate dehydrogenase, or combinations thereof, to form an in vitro cell-free reaction; and incubating the in vitro cell-free reaction to synthesize the compound.

In various embodiments of any of the above aspects, the enzyme and the ATP regeneration system are obtained from the separate sources. The sources may be independently selected from, a cell-free extract, an in vitro reaction, or combinations thereof. In various embodiments of any of the above aspects, the in vitro cell-free system further includes the compound that is synthesized in vitro (e.g., in various embodiments shikimic acid is synthesized in vitro). In various embodiments of any of the above aspects, the ATP regeneration system is a photosynthetic ATP regeneration system. In various embodiments of any of the above aspects, the ATP regeneration system is isolated from a plant, an alga, or a cyanobacterium. In various embodiments of any of the above aspects, the ATP regeneration system contains a thylakoid membrane. In various embodiments of any of the above aspects, the ATP regeneration system contains a chloroplast.

In various embodiments of any of the above aspects, the ATP regeneration system contains an ATP synthase, a cytochrome b₆-f complex, a plastocyanin, and a photosystem 1 (PS1) complex. In specific embodiments, the PS1 complex comprises the polypeptides PsaA-PsaS and the molecules chlorophyll 700, phylloquinone, Fe₄S₄, and carotenoid; and the cytochrome b₆-f complex comprises cytochrome b₆, cytochrome f, iron-sulfur protein, cytochrome b₆-f subunit IV, and the molecules heme b_{L}, heme b_{H}, heme c, and Fe₂S₂.

In various embodiments of any of the above aspects, the ATP regeneration system contains a photosystem 2 (PS2) complex and a plastoquinone. In various embodiments of any of the above aspects, the ATP regeneration system comprises an ATP synthase, a photosystem 2 (PS2) complex, a plastoquinone, and a cytochrome b₆-f complex. In specific embodiments, the PS2 complex comprises the polypeptides Cp43, Cp47, PsbO, PsbP, PsbQ, PsbE, PsbF, manganese stabilizing protein, and the molecules chlorophyll 680, pheophytin, quinone, beta carotene, and heme b559.

In various embodiments of any of the above aspects, the enzymes are polypeptides obtained from a lysate or synthesized by in vitro translation. In various embodiments of any of the above aspects, the compound is a therapeutic compound, a precursor for a therapeutic compound or a dye. In specific embodiments, the therapeutic compound is for the treatment of influenza. In specific embodiments, the compound is shikimic acid or a shikimic acid precursor. In various embodiments of any of the above aspects, the isolated composition contains the enzymes 3-deoxy-D-arabino-heptulosonate (DAHP) synthase, dehydroquinate synthase, dehydroquinate dehydratase, dehydroshikimate dehydrogenase, or combinations thereof.

In various embodiments, the in vitro cell-free system further contains protease inhibitors, amino acids, a ribosome, an RNA encoding the amino acid sequence of an enzyme that catalyzes the formation of a compound, an RNA polymerase, a DNA encoding the nucleotide sequence of an enzyme that catalyzes the formation of a compound, or combinations thereof. In various embodiments, the carbon source is a precursor for the compound. In various embodiments, the components are added in more than one step. In various embodiments, the method further comprising a step for purifying the synthesized compound.

In various embodiments, the precursor for shikimic acid is phosphoenolpyruvate (PEP), erythrose 4-phosphate (E4P), 3-deoxy-D-arabino-heptulosonate (DAHP), dehydroquinic acid, dehydroshikimic acid, or combinations thereof.

In various embodiments, the in vitro cell-free reaction further involves providing a component including protease inhibitors, amino acids, a ribosome, an RNA encoding the amino acid sequence of an enzyme that catalyzes the formation of a compound, an RNA polymerase, a DNA encoding the nucleotide sequence of an enzyme that catalyzes the formation of a compound, or combinations thereof.

In various embodiments, the ATP regeneration system or in vitro cell-free system contains pyruvate kinase (PK). In various embodiments, the ATP regeneration system or in vitro cell-free system further contains phosphoglucose isomerase, phosphofructokinase (PKK-1), frustose bisphosphate aldolase, triosephosphate isomerase (TPI), glyceraldehyde phosphate dehydrogenase (GADPH), phosphoglycerate kinase (PGK), phosphoglycerate mutase (PGM), and enolase. In various embodiments, the ATP regeneration system or in vitro cell-free system further contains hexokinase (HK). In various embodiments, the energy source includes glucose, glucose 6-phosphate, phosphoenolpyruvate, or combinations thereof.

Other features and advantages of the invention will be apparent from the detailed description, and from the claims.

### BRIEF DESCRIPTION OF THE DRAWINGS

FIG. 1 is a schematic depicting a system for the production of a compound in which an ATP regeneration system is coupled to components of an enzymatic pathway to synthesize a compound of interest.

FIG. 2 is a schematic depicting the photosynthetic pathway and the generation of adenosine triphosphate (ATP) and reduced nicotinamide adenine dinucleotide phosphate (NADPH)

FIG. 3 is a schematic depicting the shikimic acid biosynthetic pathway.

FIG. 4 is a schematic depicting a system for the production of shikimic acid involving ATP produced by a photosynthetic ATP regeneration system.

### DETAILED DESCRIPTION OF THE INVENTION

The invention features compositions and methods that are useful for the production of compounds that are difficult to synthesize on large scale, e.g., industrial or commercial scales. The production of such compounds may be cost-prohibitive or be incompatible with production methods, e.g., cytotoxicity in cell-based production methods. The invention is based on the discovery that in a cell free system an adenosine triphosphate (ATP) regeneration system may be coupled to an enzyme or pathway of enzymes, which require ATP, to synthesize a compound of interest. In particular embodiments, the ATP regeneration system is a cyclical cell-free photophosphorylation system, e.g., a photosynthetic ATP regeneration system. Energy provided in the form of light energy, e.g., solar energy, is provided for cell-free photophosphorylation systems to generate ATP. The system is particularly advantageous for the production of complex therapeutic compounds or precursors of those compounds, e.g., shikimic acid.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “adenosine triphosphate regeneration system” or “ATP regeneration system” refers to a system for producing adenosine triphosphate. In one embodiment, adenosine triphosphate (ATP) is generated from adenosine diphosphate (ADP) and an inorganic phosphate. In a specific embodiment, the ATP regeneration system comprises an ATP synthase, a cytochrome b₆-f complex, a plastocyanin, and a photosystem 1 (PS1) complex. In another specific embodiment the ATP regeneration system comprises an ATP synthase, a photosystem 2 (PS2) complex, a plastoquinone, and a cytochrome b₆-f complex. Components of the photosynthetic ATP regeneration system, including polypeptides and their cofactors, are known in the art (see, e.g., Alberts et al., Molecular Biology of the Cell, 2002; Berg et al., Biochemistry, 2002; Lodish et al., Molecular Cell Biology, 1999).

As used herein, “chloroplast” refers to organelles found in plant cells and eukaryotic algae that conduct photosynthesis. Chloroplasts absorb light and use it in conjunction with water and carbon dioxide to produce carbohydrates (e.g., for energy and biomass production in green plants). Chloroplasts are present in algae and green plants, e.g. Viridiplantae, Viridiphyta, or Chlorobionta. Chloroplasts convert light energy to potential chemical energy in the form of ATP and reduce NADP to NADPH through photosynthesis.

As used herein, “in vitro reaction” refers to a reaction performed in a controlled environment (e.g., an experimental environment or an environment outside a living organism).

As used herein, “cell-free” refers to a non-living system, e.g., in vitro or ex vivo systems containing cellular components. Sources for the components of cell-free systems include cell extracts and lysates. Cell-free systems are able to reconstitute cellular reactions, e.g., enzymatic and metabolic pathways.

As used herein, “same source” in reference to the claimed invention refers to a source of a component in which components from two or more sources have a common origin. As used herein, “separate source” in reference to the claimed invention refers to a source of a component in which components from two or more sources have a different origin (e.g., an external source). In reference to the claimed invention, components can be provided extrinsically (e.g., from a separate source) or from an external source (e.g., a source external to the system or the reaction).

As used herein, “fine chemicals” refers to pure, single chemical substances that are commercially produced with chemical reactions into highly specialized applications. Fine chemicals produced can be categorized into active pharmaceutical ingredients and their intermediates, biocides, and speciality chemicals for technical applications. The production of fine chemicals is generally more expensive (per weight) and generates more waste compared to the production of bulk chemicals, which are produced in massive quantities by standardized reactions. Fine chemicals can be produced in industrial quantities unlike research chemicals, which are produced only in the laboratory.

As used herein, the terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified.

As used herein, “photophosphorylation” refers to a light-dependent reaction that results in the production of adenosine triphosphate (ATP), e.g., using solar energy. In photophosphorylation, light energy is used to create a high-energy electron donor and a lower-energy electron acceptor. Electrons then move from donor to acceptor through an electron transport chain. The redox reactions are coupled to a system for the production of ATP.

As used herein, “photosynthesis” refers to a metabolic pathway that converts light energy into chemical energy. In photosynthesis, “light dependent reactions” or “photosynthetic reactions” produce adenosine triphosphate (ATP) and reduced nicotinamide adenine dinucleotide phosphate (NADPH) by photophosphorylation. In the light dependent reactions, light energy is used to excite an electron which generates a series of redox reactions in an electron transport chain that produce a transmembrane electrochemical potential gradient, e.g., across a thylakoid membrane. An ATP synthase polypeptide complex is powered by the transmembrane electrochemical potential gradient, in the form of a proton gradient. The ATP synthase complex generates ATP from ADP and an inorganic phosphate. Components of the photosynthetic pathway involved in the light dependent reactions include ATP synthase, cytochrome b₆-f complex, plastocyanin, plastoquinone, photosystem 1 (PS1) complex, and photosystem 2 (PS2) complex. In “light independent reactions” or “dark reactions” chemical reactions convert carbon dioxide and other compounds into glucose, e.g., Calvin-Benson cycle. Photosynthetic pathways may be found, for example, in plants, algae, bacteria, e.g., cyanobacteria.

As used herein, “photosystem” refers to a complex of polypeptides and co-factors that are involved in photosynthesis. A photosystem transfers light energy and transfers electrons through an electron transport chain. “Photosystem 1” or “PS1” refers to a complex of polypeptides and co-factors including the polypeptides PsaA, PsaB, PsaC, Ps-PsaS and the molecules chlorophyll 700, phylloquinone, Fe₄S₄, and carotenoid; and the cytochrome b₆-f complex comprises cytochrome b₆, cytochrome f, iron-sulfur protein, cytochrome b₆-f subunit IV, and the molecules heme b_{L}, heme b_{H}, heme c, and Fe₂S₂. “Photosystem 2” or “PS2” refers to a complex of polypeptides and co-factors including the polypeptides Cp43, Cp47, PsbO, PsbP, PsbQ, PsbE, PsbF, manganese stabilizing protein, and the molecules chlorophyll 680, pheophytin, quinone, beta carotene, and heme b559.

As used herein, “thylakoid” refers to a membrane-bound compartment inside chloroplasts and cyanobacteria involved in photosynthesis.

As used herein, “thylakoid membrane” refers to a membrane in the thylakoid that contains or physically interacts with the components of the photosynthetic pathway, including the PS1 and PS2 complexes and the polypeptides containing the electron transport pathway.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. The amino acid sequences of the polypeptides described herein and the nucleic acid sequences encoding the polypeptide sequences described herein are known in the art and can be found in public sequence databases (e.g., NCBI Genbank).

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### Adenosine Triphosphate (ATP) Production in a Cell-Free System.

In various embodiments, the invention provides compositions and methods for the production of adenosine triphosphate (ATP) in a cell-free reaction system. In biological systems, ATP is generated from adenosine diphosphate and an inorganic phosphate (Pi). In particular, the invention provides photosynthetic ATP regeneration systems obtained from biological organisms that perform photosynthetic reactions, including plants, algae, and bacteria, e.g., cyanobacteria. The invention advantageously provides a self-regulating, continuous ATP regeneration system. Additionally, reduced nicotinamide adenine dinucleotide phosphate (NADPH) is generated through the photoreactions. It is a goal of the invention to extend cell-free technology to plant systems.

### Preparation of a Cellular Extract

In one embodiment of the invention, a cellular extract of a photosynthetic organism is provided containing the components of the photosynthetic pathway. Typically, the components of the photosynthetic pathway are present in the thylakoid membranes or photosynthetic bacterial membranes, e.g., from cyanobacteria. Thylakoid membranes may be obtained from any plant, algae, or photosynthetic bacteria. Typically, the photosynthetic organism is grown under conditions favorable for their growth prior to extraction of thylakoid membranes. Large quantities of cells of a photosynthetic organism may be grown in a fermenter or bioreactor according to methods known in the art for growing cells. Preferably, the methods and conditions for extracting thylakoid membranes leave the membrane associated components of the photosystem intact. Cyclic and non-cyclic photosynthetic machinery of plant cells used to catalyze ATP production through ferredoxin light activation and NADP+ reduction are preserved ex vivo in cell-free systems (Dani et al., Biochim. Biophys. Acta (2005) 1669: 43-52; Ono et al., Biochim. Biophys. Acta (1978) 502: 477-485). Thylakoid membranes may also be isolated as whole chloroplasts.

To obtain cellular extract for ATP regeneration, cyanobacteria, e.g. Anacystis nidulans, containing thylakoid membrane are lysed. This cell extract can be generally obtained by rupturing the cells, e.g. with a French press, in a buffer containing salts for solubilizing protein components. The homogenate may be further processed to allow separation of the membrane-bound organelles and cell membranes from the cell wall, e.g., filtering and/or centrifuging the lysate at low speed (e.g., 3K-5K rpm), and using the filtrate or supernatant. The extract can also be prepared from a green plant (Viridiplantae, Viridiphyta or Chlorobionta). Methods and conditions for obtaining lysates from cells, including plant cells, are known in the art. To isolate an extract containing a photosynthetic ATP regeneration system from a plant, plant tissue containing chloroplasts are collected and homogenized under conditions that result in cell lysis. The plant extract may also be cleared by a filtration step or low speed centrifugation step. Following preparation of the extract, a dialysis step may be employed to adjust the buffer composition or conditions, e.g., ionic, pH, etc. The extract may be further flash frozen using liquid nitrogen and stored at −80° C. until needed for ATP synthesis. The cellular extract used to regenerate ATP may be a crude lysate or a partially or substantially purified fraction containing thylakoid membranes. Methods of determining whether a fraction contains thylakoid membranes are known in the art and may include determining the density of the fraction or the enrichment of a marker, e.g., proteins in the photosynthetic pathway, photosynthetic pigments, or photosynthetic co-factors. Such methods may involve routine laboratory procedures, including sodium dodecyl sulfate polyacrylamide gel electrophoresis (SDS-PAGE), Western blot analysis, spectroscopic analysis, and chromatography. The extract for ATP regeneration may be provided in a fresh or frozen form, and may further be formulated into a reaction mix suitable for use with polypeptide synthesis. Such extracts are obtained by any of the methods known in the art for the purpose of cell-free protein synthesis.

Additional genetic modifications may also be made to the plant or microbial strain for obtaining cellular extracts. Methods for transfection or transformation of cyanobacteria are known in the art, e.g., Daniell et al., Proc. Natl. Acad Sci, USA (1986) 83:2546-2550. Transgenic plants with desirable properties can be created by on skilled in the art, for example by the introduction of recombinant DNA molecules. Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Several standard methods are available for introduction of the vector into a plant host, thereby generating a transgenic plant. These methods include (1) Agrobacterium-mediated transformation (A. tumefaciens or A. rlzizogenes) (see, e.g., Lichtenstein and Fuller In: Genetic Engineering, vol 6, PWJ Rigby, ed, London, Academic Press, 1987; and Lichtenstein, C. P., and Draper, J. In: DNA Cloning, Vol II, D. M. Glover, ed, Oxford, IRI Press, 1985)), (2) the particle delivery system (see, e.g., Gordon-Kamm et al., Plant Cell 2: 603 (1990); or BioRad Technical Bulletin 1687, supra), (3) microinjection protocols (see, e.g., Green et al., supra), (4) polyethylene glycol (PEG) procedures (see, e.g., Draper et al., Plant Cell Physiol. 23: 451, 1982; or e.g., Zhang and Wu, Theor. Appl. Genet. 76: 835, 1988), (5) liposome-mediated DNA uptake (see, e.g., Freeman al., Plant Cell Physiol. 25: 1353, 1984), (6) electroporation protocols (see, e.g., Gelvin et al., supra; Dekeyser et al., supra; Fromm et al., Nature 319: 791, 1986; Sheen Plant Cell 2: 1027, 1990; or Jang and Sheen Plant Cell 6: 1665, 1994), and (7) the vortexing method (see, e.g., Kindle supra).

### ATP Production

Light energy, i.e., photons, is provided to the ATP regeneration system to produce ATP from ADP and inorganic phosphate. Without being bound to any particular theory, in the light reaction of photosynthesis one molecule of the pigment (e.g., chlorophyll in a green plant) absorbs one photon and loses one electron. The electron is transferred to a modified form of chlorophyll called pheophytin, which transfers the electron to a quinone molecule, allowing the flow of the electron down an electron transport chain that leads to the ultimate reduction of NADP to NADPH. Flow of electrons in the electron transport chain creates a proton gradient across the chloroplast membrane. Either non-cyclic or cyclic electron transport may be used to create the proton gradient. Non-cyclic electron transport involves photosystems 1 and 2, where photosystem 1 accepts electrons from plastocyanin and reduces NADP. The photosystem 2 complex generates a proton in the thylakoid lumen when photons are absorbed by the pigment and a water molecule becomes oxidized. Additionally, transport of an electron from the photosystem 2 complex to plastoquinone to the b₆f complex generates a proton in the thylakoid lumen. Cyclic electron transport involves the excitation of electrons in photosystem 1 and their transfer to cytochrome b₆-f complex. The dissipation of the proton gradient is used by ATP synthase for the concomitant synthesis of ATP. The chlorophyll molecule regains the lost electron from a water molecule through a process called photolysis, which releases a dioxygen (O₂) molecule.

The wavelength of light used to generate ATP from the cellular extract is based on the action spectrum, which depends on the type of accessory pigments present. For example, in green plants, the action spectrum resembles the absorption spectrum for chlorophylls and carotenoids with peaks for violet-blue and red light. In red algae, the action spectrum overlaps with the absorption spectrum of phycobilins for blue-green light, which allows these algae to grow in conditions that filter out the longer wavelengths used by green plants. The non-absorbed part of the light spectrum provides photosynthetic organisms their color (e.g., green plants, red algae, purple bacteria) and is the least effective for photosynthesis in the respective organisms. Based on the action spectrum absorption spectrum, it would be apparent from one skilled in the art to select an appropriate wavelength of light in the photosynthetic ATP regeneration system.

Operating ranges of the ATP regeneration system include those between 4-60° C. and pH 2-12. Preferably, the reaction is maintained in the range of pH 5-10 and a temperature of 20°-50° C., and more preferably, in the range of pH 6-9 and a temperature of 20°-35° C. The conditions may be changed, e.g., cycled, within this range to permit changes in the reaction conditions. Conditions typical for cell growth (e.g., 30-37° C., 4<pH<9, CO₂<1%) may also be used, although it is an advantage of the system that it is not bound by conditions for maintaining cell viability. The ATP produced may be isolated and stored or used to provide energy to ATP-dependent reactions, e.g., coupling in a cell-free system. The cellular extract of the invention may also comprise protease inhibitors. If the in vitro synthesis of polypeptides is desired in the extract, amino acids, ribosomes, RNA encoding the amino acid sequence of a polypeptide, RNA polymerase, a DNA encoding the nucleotide sequence of polypeptide, or any combination of these factors may be added to the cellular extract. It is expected the ATP regeneration system can be scaled and/or may be affected by kinetic limitations. Experimentation to determine these parameters to operate the system is routine and is not undue.

### Carbon Sequestration

In another embodiment of the invention, carbon dioxide can be fixed into organic compounds in the ATP regeneration system. Carbon fixation is a process found in autotrophs (organisms that produce their own food), usually driven by photosynthesis, whereby carbon dioxide is changed into organic materials, e.g., glyceraldehyde-3-phosphate. Carbon fixation in plants generally involves the dark/light-independent reactions, e.g., Calvin Cycle. The enzymes of the dark/light-independent reactions may also be present in the extracts used in the ATP regeneration system. Thus, an advantage of the ATP regeneration system is that it can produce organic materials, which can be isolated or used as starting material for additional reactions.

Glyceraldehyde-3-phosphate (G3P) can also be generated in the cell-free system by carbon fixation reactions, e.g., the Calvin Cycle pathway. Enzyme components of the Calvin Cycle include Ribulose-1,5-bisphosphate carboxylase/oxygenase (RuBisCO), phosphoglycerate kinase, and glyceraldehyde-3-phosphate dehydrogenase. The production of G3P requires Ribulose-1,5-bisphosphate, ATP and NADPH, which is used as an electron donor for carbon fixation. Glyceraldehyde-3-phosphate is an intermediate in several central metabolic pathways of all organisms, including the assembly of hexoses such as glucose. An advantage of the cell-free system is the elimination of the need for expensive hexose energy sources. Additionally, the production of hexoses is an advantage of the cell-free system, when if hexoses are the starting reactants for the production of a compound of interest.

### Production of a Compound by Coupling to ATP Regeneration System.

In one embodiment, the invention provides the integration of photosynthetic systems with cell-free synthesis of a compound of interest. Compositions and methods are provided to synthesize compounds enzymatically in a cell-free reaction system using the ATP provided by an ATP regeneration system. The enzymes or enzyme pathways useful for synthesizing compounds include those involved in cellular biochemical or metabolic reactions. An advantage of the cell-free system is that the enzymes for synthesizing a compound can be exogenously added (i.e., from a separate source, including in vitro translation reactions, cell lysates). Sources for the enzymes include microbial and eukaryotic sources and/or naturally occurring and recombinant sources and/or crude or purified sources. Recombinant sources may provide a useful genetic alteration, including providing a desired altered activity or function to an enzyme.

The invention addresses several problems associated with existing cell-free reactions, including the need for exotic energy schemes to produce ATP in situ non-closed loop system; ATP synthase limitation (e.g., free phosphate equilibrium shuts down synthesis); and the need to introduce carbon source directly (e.g., hexoses). Although cell-based biotechnology methods have proven useful, such methods are limited by conditions of cell viability, e.g., the narrow operating ranges needed for cell growth (30-37° C., 4<pH<9, CO₂<1%). Wide ranges of concentrations, temperatures, pH can be used in the cell-free system. When using whole or intact cells, all energy cannot be directed towards synthesis, and energy is used in essential cell functions, including cell growth and maintenance (e.g., cytoskeletal maintenance) and cell duplication, (e.g., DNA replication). Advantages of the invention over the cells, which are enclosed by a cell membrane, are the ability to introduce exogenous factors for synthesis (e.g., enzymes, co-factors, DNA constructs to direct synthesis of non-natural molecules); and in vitro synthesis rates are not limited to cell membrane/cell wall permeability. An additional advantage of the cell-free system of the invention in comparison to cell-based methods, is the elimination of the need for expensive hexose energy sources to support cell growth ATP production. Furthermore, a feature of the cell-free system is the ability to produce carbon sources for energy and/or enzymatic precursors through carbon fixation reactions, e.g., Calvin cycle reactions.

Compounds of interest that can be synthesized in the invention include therapeutic compounds and/or their precursors, fine chemicals, monomers, industrial chemicals, fuel replacement products. In contrast to cell-based methods, the cell-free system of the invention can produce highly cytotoxic compounds (i.e., toxic to cellular systems), including biocides, anticancer compounds, and antibiotics. Additionally, the cell-free provides advantages for the production of carbon-neutral synthetic products, including fuel replacement products (e.g. butanol, butanediol), consumer products (e.g., plastics), and hydrocarbon-based fine chemicals and drug precursors. Hydrocarbons inhibit cell growth at high concentrations and cannot be produced in significant quantities by cell-based methods (Table 1).

TABLE 1
Production of in bacterial, fungal systems severely inhibits cell growth.

| | Energy Density | |
|---|---|---|
| Molecule | (MJ/L) | Inhibition Concentration |
| Ethanol | 19.6 | 5% in E. Coli/15% in yeast |
| 1-butanol | 29.2 | 1.3% in E. coli |
| 1-pentanol | 30.3 | 0.25% in E. Coli |
| 1-decanol | 31.9 | 0.000236% in E. Coli |
| Gasoline | 32 | N/A |

The cell-free system of the invention provides the ability to synthesize carbohydrate chains and synthesis of C₃-Cₙ hydrocarbons, e.g. using enzymes in bacterial lysates. For the synthesis of long chain hydrocarbon, pyruvate may be used as starting material. Energy source for long hydrocarbon synthesis is provided by decarboxylation of pyruvate to acetyl-coenzyme A. Higher alcohols can be synthesized through gluconeogenesis followed by anaerobic fermentation and further reactions (e.g. aminotransferase reactions, reduction to lactate). Furthermore, in the production of fuel, kinetic and thermodynamic analysis of cell-free systems shows significant economic benefits over oil-based processes (at ˜$100/barrel).

### Source of Enzymes for Synthesis of a Compound

### Cellular Extracts Containing Enzymes

In one embodiment of the invention, a cellular extract of a bacterial strain is provided in which genetic sequences encoding at least one protein is expressed. The use of E. coli is of particular interest, where the accessory proteins are exogenous to the host cell, and may include one, two or all of polypeptides for the production of shikimic acid obtained from any suitable protein producing host, e.g. Shewanella, Chladymonas, Clostridia, etc. Additional genetic modifications may also be made to the microbial strain, for example the deletion of tonA and endA genes to protect against bacteriophage infection and stabilize DNA within the system, the deletion of proteins involved in amino acid degradation, and the like.

The polypeptides may also be isolated and purified in accordance with conventional methods of recombinant synthesis. The reaction mixture may be purified using HPLC, exclusion chromatography, gel electrophoresis, affinity chromatography, or other purification technique. The polypeptides are added to the ATP regeneration system.

The coding sequence for one or more polypeptides for the production of a compound of interest are present or introduced into the source organism, and may be present on a replicable vector or inserted into the source organism genome using methods well-known to those of skill in the art. Such vector sequences are well known for a variety of bacteria. The expression vector may further comprise sequences providing for a selectable marker, induction of transcription, etc.

The coding sequences are operably linked to a promoter sequence active in the source organism. Promoters are untranslated sequences located upstream (5′) to the start codon of a structural gene (generally within about 100 to 1000 bp) that control the transcription and translation of particular nucleic acid sequence. Promoters may be constitutive or inducible, where inducible promoters initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to protein-encoding DNA by removing the promoter from the source DNA, e.g. by PCR amplification of the sequence, etc. and inserting the isolated sequence into the vector. Both the native promoter sequence and many heterologous promoters may be used for expression, however, heterologous promoters are preferred, such as T7, as they generally permit greater transcription and higher yields. Promoters suitable for use with prokaryotic hosts include the β-lactamase and lactose promoter systems; alkaline phosphatase; a tryptophan (trp) promoter system; an arabinose promoter system; and hybrid promoters such as the tac promoter. However, other known bacterial and bacteriophage promoters are suitable. Their nucleotide sequences have been published, thereby enabling a skilled worker operably to ligate them to DNA encoding proteins.

Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

Host cells are transfected and preferably transformed with the above-described expression or cloning vectors and cultured in conventional nutrient media modified as appropriate for inducing promoters, selecting transformants, and preparing extracts as set forth in the Examples.

### Cell-Free Protein Synthesis

As used herein, “Cell-Free Protein Synthesis” refers to the cell-free synthesis of polypeptides in a reaction mix comprising biological extracts and/or defined reagents. The reaction mix will comprise at least an ATP regeneration system (including ADP and inorganic phosphate), ATP, and/or an energy source; a template for production of the macromolecule, e.g. DNA, mRNA, etc.; amino acids, and such co-factors, enzymes and other reagents that are necessary for the synthesis, e.g. ribosomes, tRNA, polymerases, transcriptional factors, etc. In one embodiment of the invention, the energy source is a homeostatic energy source. In addition to the photosynthetic ATP regeneration system, enzyme(s) that catalyze the regeneration of ATP, from high energy phosphate bonds, e.g. acetate kinase, creatine kinase, etc., a cell-free extract may also be included. Such enzymes may be present in the extracts used for translation, or may be added to the reaction mix. Such synthetic reaction systems are well-known in the art, and have been described in the literature. The cell free synthesis reaction may be performed as batch, continuous flow, or semi-continuous flow, as known in the art.

As used herein, “reaction mix” refers to a reaction mixture capable of catalyzing the synthesis of polypeptides from a nucleic acid template. The reaction mixture comprises cellular extracts cells, e.g. bacteria for plant extracts, as described above, and the synthesis is performed under anaerobic conditions. The volume percent of extract in the reaction mix will vary, where the extract is usually at least about 10% of the total volume; more usually at least about 20%; and in some instances may provide for additional benefit when provided at least about 50%; or at least about 60%; and usually not more than about 75% of the total volume.

Other salts, particularly those that are biologically relevant, such as manganese, may also be added. Potassium is generally added between 50-250 mM and ammonium between 0-100 mM. The pH of the reaction is generally run between pH 6-9. The temperature of the reaction is generally between about 20° C. and 40° C. These ranges may be extended. It has been found that synthesis of proteins may benefit from lowered reaction temperatures, where synthesis is performed at a temperature of at least about 20° C., usually at least about 23° C.; and may be about 25° C.; although conventional temperatures for synthesis are not excluded.

The synthesis may be performed for varying lengths of time, depending, in part, on whether the reaction is a batch or continuous feed. For batch reactions, the reactions may continue to accumulate protein for at least about 1 hour, usually at least about 3 hours, more usually at least about 6 hours, and may benefit from reactions time of at least about 12 hours, at least about 18 hours, at least about 24 hours, or longer, particularly where the synthesis is performed at temperatures of less than about 25° C.

If translation of mRNA coupled to in vitro synthesis of mRNA from a DNA template to produce proteins, is to be performed, the cell-free system will contain all factors required for the translation of mRNA, for example ribosomes, amino acids, tRNAs, aminoacyl synthetases, elongation factors and initiation factors. Cell-free systems known in the art include E. coli extracts, etc., which can be treated with a suitable nuclease to eliminate active endogenous mRNA.

In addition to the above components such as cell-free extract, genetic template, and amino acids, materials specifically required for protein synthesis may be added to the reaction. These materials include salts, polymeric compounds, cyclic AMP, inhibitors for protein or nucleic acid degrading enzymes, inhibitors or regulators of protein synthesis, oxidation/reduction adjusters, non-denaturing surfactants, buffer components, spermine, spermidine, etc.

The salts preferably include potassium, magnesium, ammonium and manganese salts of acetic acid, glutamic acid, or sulfuric acid, and some of these may have other amino acids as a counter anion. The polymeric compounds may be polyethylene glycol, dextran, diethyl aminoethyl dextran, quaternary aminoethyl and aminoethyl dextran, etc. The oxidation/reduction adjuster may be dithiothreitol, ascorbic acid, glutathione and/or their oxides. Also, a non-denaturing surfactant such as Triton X-100 may be used at a concentration of 0-0.5 M. Spermine and spermidine and/or putrescine may be used for improving protein synthetic ability, and cAMP may be used as a gene expression regulator.

The amount of protein produced in a translation reaction can be measured in various ways. One method relies on the availability of an assay that measures the activity of the particular protein being translated. Examples of assays for measuring protein activity are the methyl viologen assay described in the examples. These assays measure the amount of functionally active protein produced from the translation reaction. Activity assays will not measure full length protein that is inactive due to improper protein folding or lack of other post translational modifications necessary for protein activity.

A method of measuring the amount of compound produced in a combined in vitro transcription and translation reactions is to perform the reactions using a known quantity of radiolabeled amino acid such as ³⁵S-methionine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

### Shikimic Acid Synthesis

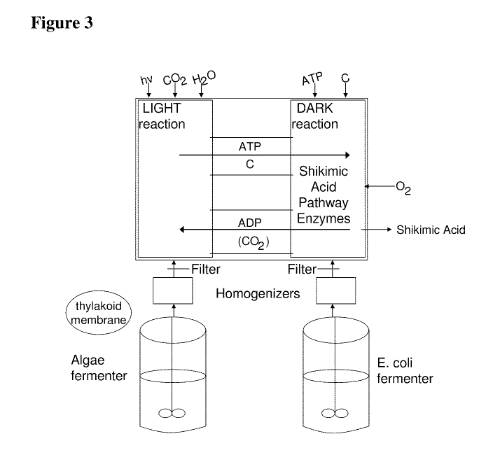

The biosynthesis of shikimic acid is known in the art, e.g., as shown in FIG. 3. A cell extract may be prepared from a bacteria or other cell that produces the enzymes involved in shikimic acid biosynthesis. In the shikimic acid synthesis pathway, phosphoenolpyruvate and erythrose-4-phosphate react to form 3-deoxy-D-arabinoheptulosonate-7-phosphate (DAHP), in a reaction catalysed by the enzyme DAHP synthase. DAHP is then transformed to 3-dehydroquinate (DHQ), in a reaction catalysed by DHQ synthase. Although this reaction requires NAD as a cofactor, the enzymic mechanism regenerates it, resulting in the net use of no NAD. DHQ is dehydrated to 3-dehydroshikimate by the enzyme dehydroquinase, which is reduced by to shikimic acid by the enzyme shikimate dehydrogenase, which uses NADPH as a cofactor. Additionally, shikimic acid may be produced from any of the intermediates present in the pathway and the enzymes that lead. Additionally, it would be apparent to one of skill in the art to adjust the levels and activities of the enzymes to favor the production of a compound in the pathway or to reduce the production of other intermediates in the pathway.

Preferably, the reaction is maintained in the range of pH 5-10 and a temperature of 20°-50° C., and more preferably, in the range of pH 6-9 and a temperature of 20°-35° C. Although photosynthetic ATP regeneration system is described, any ATP regeneration system may be used, e.g., the ATP regeneration system described in PCT Patent Publication No. WO2000055353A1.

A method of measuring the amount of compound produced in a cell-free system (combining in vitro ATP regeneration and compound synthesis reactions) is to perform the reactions using a known quantity of a radiolabeled precursor (e.g., one containing ³⁵S, ¹⁴C, ³H) and measuring production of compound based on the incorporation of the radiolabel.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

### Reactor Design

In one embodiment, the invention provides the assembly of algae/plant-origin thylakoid membranes in a photoreactor. In another the embodiment, the invention provides the assembly of a reactor in which photosynthetic reactions are coupled to reactions for the synthesis of a compound of interest. The reactions may be large scale, small scale, or may be multiplexed to perform a plurality of simultaneous syntheses. It is expected that ATP regeneration reactions and the reactions for producing a molecule of interest can be scaled and may be affected by kinetic limitations. For example, methods for scaling in vitro methods are described in U.S. Pat. No. 7,341,852B2. However, experimentation to determine process scale-up and process optimization is routine and is not undue. Large scale fermentors may be used for the growth of cells for extracts. The conditions under which the reactor operates may be temporally altered to accommodate a particular step (e.g., ATP regeneration; cell-free protein). Additional reagents may be introduced to prolong the period of time for active synthesis. Synthesized product is usually accumulated in the reactor, and then is isolated and purified according to the usual methods for protein purification after completion of the system operation. In some cases, enzyme activity may be determined and used without purification.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

All patents and publications mentioned in this specification are herein incorporated by reference to the same extent as if each independent patent and publication was specifically and individually indicated to be incorporated by reference. The following patents and patent applications may disclose related subject matter, and each is incorporated by reference in their entirety: U.S. Pat. No. 7,312,049, U.S. Pat. No. 7,341,852, U.S. Pat. No. 6,168,931, US20070154983, US20060281148, US20050054044, US20040209321, US20030113778, US20020058303, WO2008088884, WO2008066583, WO2008002661, WO2008002673, WO2008002663, WO2007053655, WO2005098048. In case of conflict, the present specification, including definitions, will control. In addition, the materials, methods, and examples are illustrative only and not intended to be limiting. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXAMPLE

The present invention is described by reference to the following Examples, which are offered by way of illustration and are not intended to limit the invention in any manner. Standard techniques well known in the art or the techniques specifically described below were utilized.

### Example 1

### Production of Shikimic Acid by Coupling to Photosynthetic ATP Regeneration System

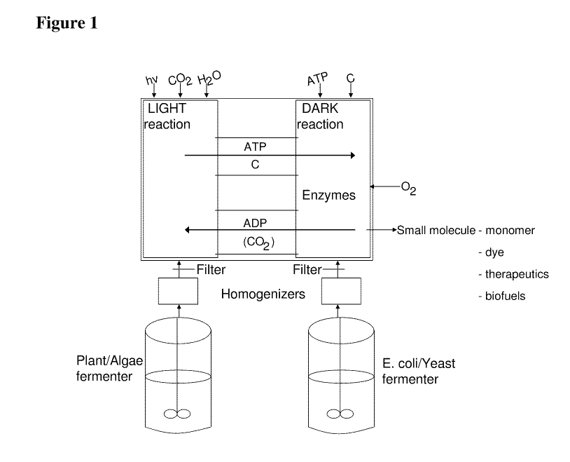

The invention provides compositions and methods for the production of adenosine triphosphate (ATP) in a cell-free system and for the production of compounds by enzymes or pathways of enzymes in a cell-free system. An example of a generic cell-free system is provided in FIG. 1. ATP is produced by an ATP regeneration system which catalyzes the formation of ATP from adenosine diphosphate (ADP) and an inorganic phosphate. ATP regeneration systems include photosynthetic ATP regeneration systems of plants and other photosynthetic organisms. Enzymes or pathways of enzymes that require ATP may be combined with the ATP regeneration system to produce compounds of interest, e.g., shikimic acid. The study assesses the ability of isolated thylakoid fractions to generate ATP in vitro. The study assesses the rate and production of ATP and the production of shikimic acid in a cell-free system. It is a goal of the invention to extend cell-free technology to photosynthetic systems.

A photosynthetic ATP regeneration system is isolated from cyanobacteria, e.g., Anacystis nidulans. Preferably, the cyanobacteria are grown under conditions favorable for growth prior to the extraction of the cell lysate. The cell lysate may be used as extracted, or a step may be used to clear unlysed cells and cytoskeletal debris from the lysate, e.g., a filtration step. The cyanobacteria cell lysate contains thylakoid membranes. The thylakoid membranes may be purified further by methods known to one skilled in the art before subsequent use.

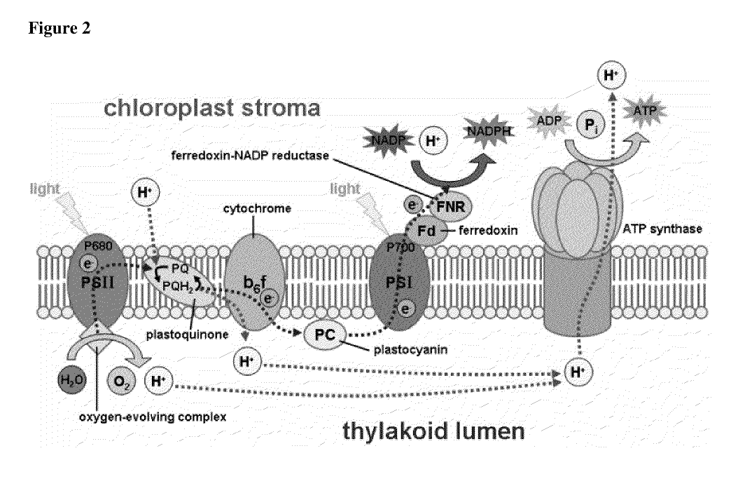

ATP and NADPH are generated through photoreactions in the cell-free system (FIG. 2). ADP and inorganic phosphate are present in the cell-free system. ADP and inorganic phosphate may be exogenously added to the cell-free system, e.g., from a purified source or another biological source. NADP is also present in the cell-free system, and additional NADP may be exogenously added to the cell-free system. The system is exposed to light of wavelength between about 530-720 nm. Preferably solar energy is used, but any source of light having wavelengths absorbed by the photosynthetic pigments may be used. Light absorbed by the photosynthetic pigments is used to reduce a water molecule resulting in the generation of an electron, which is transferred through the electron transport chain, which is present in the thylakoid membrane. Components of the electron transport chain include the polypeptides, protein complexes, and co-factors (e.g., a cytochrome b₆-f complex, a plastocyanin, and a photosystem 1 complex, photosystem 2 (PS2) complex, and a plastoquinone) which act as electron donors and electron acceptors in a set of biochemical reactions to transfer of protons across a membrane. The electron transport pathway generates a transmembrane electrochemical potential gradient in the form of a proton gradient. The proton gradient provides energy to an ATP synthase, which catalyzes the formation of ATP from ADP and a phosphate. The amount of ATP produced is compared to that by cultured plant cells in vitro by methods known in the art, e.g., HPLC or biochemical assays. Thus ATP production can be demonstrated to be at near-in vivo rates in cell-free systems.

Glyceraldehyde-3-phosphate (G3P) may also be generated in the cell-free system by carbon fixation reactions, e.g., the Calvin Cycle pathway. Enzyme components of the Calvin Cycle include Ribulose-1,5-bisphosphate carboxylase/oxygenase (RuBisCO), phosphoglycerate kinase, and glyceraldehyde-3-phosphate dehydrogenase. The production of G3P requires Ribulose-1,5-bisphosphate, ATP and NADPH, which is used as an electron donor for carbon fixation. Glyceraldehyde-3-phosphate is an intermediate in several central metabolic pathways of all organisms, including the assembly of hexoses such as glucose. An advantage of the cell-free system is the elimination of the need for expensive hexose energy sources.

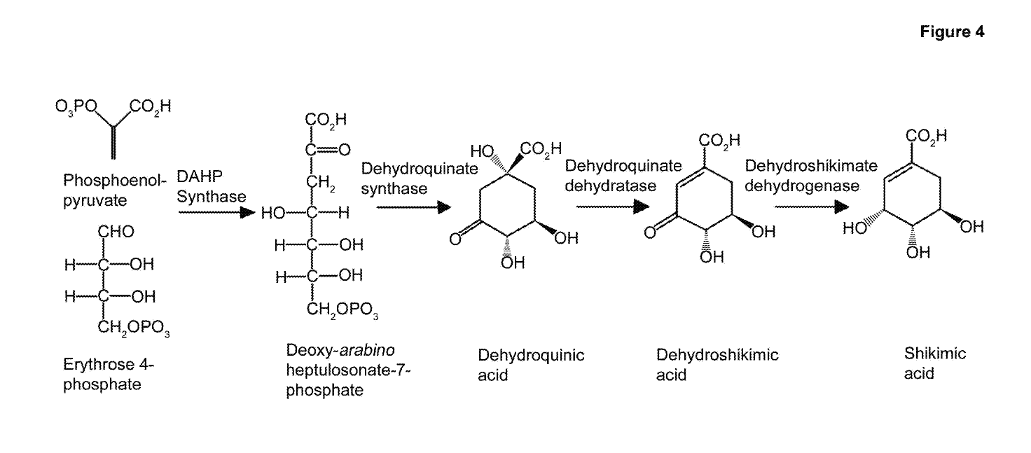

The photosynthetic cell-free system can be coupled to the shikimic acid pathway, thereby providing an energy source for the ATP dependent reactions (FIG. 3). The biosynthesis of shikimic acid is known in the art, e.g., as shown in FIG. 4. A cell extract is produced from bacteria containing the enzymes involved in shikimic acid biosynthesis. Phosphoenolpyruvate and erythrose-4-phosphate react to form 3-deoxy-D-arabinoheptulosonate-7-phosphate (DAHP), in a reaction catalysed by the enzyme DAHP synthase. An additional advantage of the cell-free system is the production of carbon compounds through carbon fixation reactions and subsequent reactions, e.g., glycolysis, can yield compounds for shikimic acid synthesis, e.g., phosphoenolpyruvate (PEP) DAHP is then transformed to 3-dehydroquinate (DHQ), in a reaction catalysed by DHQ synthase. Although this reaction requires NAD as a cofactor, the enzymic mechanism regenerates it, resulting in the net use of no NAD. DHQ is dehydrated to 3-dehydroshikimate by the enzyme dehydroquinase, which is reduced by to shikimic acid by the enzyme shikimate dehydrogenase, which uses NADPH as a cofactor.

The enzymes are typically provided in a cell-free lysate obtained from bacteria that contain the enzymes for shikimic acid biosynthesis. The bacteria are grown under conditions favorable for bacterial growth, e.g., log phase, and/or conditions favorable for the production of the enzymes involved in shikimic acid biosynthesis. Recombinant bacteria may be used containing the enzymes or modified variants of the enzymes for shikimic acid biosynthesis. Additionally, shikimic acid may be provided by enzymes produced by in vitro translation or transcription/translation reactions. It will be apparent to one of skill in the art that, depending on the precursor provided, not all the enzymes need be provided for the production of shikimic acid and that the levels of the enzymes in the cell-free system may be adjusted to favor the over the production of other intermediates and products. Thus, the reaction scheme for shikimic acid synthesis is demonstrated in a cell-free systems.

A method of measuring the amount of shikimic acid produced in a cell-free system can be shown by performing the reactions using a known quantity of a radiolabeled precursor (e.g., one containing ³⁵S, ¹⁴C, ³H) and measuring production of compound based on the incorporation of the radiolabel. Methods of determining the presence of compounds such as shikimic acid are known in the art and involve routine laboratory procedures, including spectroscopic assays and chromatography, e.g., high pressure liquid chromatography (HPLC).

The shikimic acid produced may be isolated and stored. The shikimic acid may be used in the production of a therapeutic antiviral compound, e.g., TAMIFLU. If the shikimic acid is for the production of a therapeutic agent, it is purified at least to the grade of a fine chemical. Methods for the purification of shikimic acid are known in the art and may involve routine laboratory procedures for the purification of compounds, e.g., precipitation, chemical extraction, size exclusion chromatography, ion exchange chromatography, HPLC, etc.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated composition comprising an enzyme that catalyzes the formation of a compound and an adenosine triphosphate (ATP) regeneration system, wherein the enzyme and the ATP regeneration system are from the same source or separate sources, and wherein at least one source is a cell-free extract.

2. The isolated composition of claim 1, wherein the separate sources are selected from the group consisting of a cell-free extract, an in vitro reaction, and combinations thereof.

3. The isolated composition of claim 1, wherein the ATP regeneration system is a photosynthetic ATP regeneration system.

4. The isolated composition of claim 1, wherein the ATP regeneration system is isolated from a plant, an alga, or a cyanobacterium.

5. The isolated composition of claim 4, wherein the ATP regeneration system comprises a thylakoid membrane.

6. The isolated composition of claim 4, wherein the ATP regeneration system comprises a chloroplast.

7. The isolated composition of claim 1, wherein the ATP regeneration system comprises an ATP synthase, a cytochrome b₆-f complex, a plastocyanin, and a photosystem 1 (PS1) complex.

8. The isolated composition of claim 7, wherein the PS1 complex comprises the polypeptides PsaA-PsaS and the molecules chlorophyll 700, phylloquinone, Fe₄S₄, and carotenoid; and the cytochrome b₆-f complex comprises cytochrome b₆, cytochrome f, iron-sulfur protein, cytochrome b₆-f subunit IV, and the molecules heme b_{L}, heme b_{H}, heme c, and Fe₂S₂.

9. The isolated composition of claim 7, further comprising a photosystem 2 (PS2) complex and a plastoquinone.

10. The isolated composition of claim 1, wherein the ATP regeneration system comprises an ATP synthase, a photosystem 2 (PS2) complex, a plastoquinone, and a cytochrome b₆-f complex.

11. The isolated composition of claim 9, wherein the PS2 complex comprises the polypeptides Cp43, Cp47, PsbO, PsbP, PsbQ, PsbE, PsbF, manganese stabilizing protein, and the molecules chlorophyll 680, pheophytin, quinone, beta carotene, and heme b559.

12. The isolated composition of claim 1, wherein enzymes comprise polypeptides obtained from a lysate or synthesized by in vitro translation.

13. The isolated composition of claim 1, wherein the compound is a therapeutic compound, a precursor for a therapeutic compound or a dye.

14. The isolated composition of claim 13, wherein the compound is shikimic acid.

15. The isolated composition of claim 13, wherein the isolated composition comprises an enzyme selected from the group consisting of 3-deoxy-D-arabino-heptulosonate (DAHP) synthase, dehydroquinate synthase, dehydroquinate dehydratase, dehydroshikimate dehyrdogenase, and combinations thereof.

16. An in vitro cell-free system for the synthesis of a compound comprising:
a carbon source, a phosphate source, water, an enzyme that catalyzes the formation of a compound, an energy source, adenosine diphosphate (ADP), and an adenosine triphosphate (ATP) regeneration system.

17. The in vitro cell-free system of claim 16, further comprising the compound that is synthesized in vitro.

18. The in vitro cell-free system of claim 16, wherein the ATP regeneration system is a photosynthetic ATP regeneration system.

19. The in vitro cell-free system of claim 16, wherein the ATP regeneration system is isolated from a plant, an alga, or a cyanobacterium.

20. The in vitro cell-free system of claim 19, wherein the ATP regeneration system comprises a thylakoid membrane.

21. The in vitro cell-free system of claim 19, wherein the ATP regeneration system comprises chloroplasts.

22. The in vitro cell-free system of claim 16, wherein the ATP regeneration system comprises an ATP synthase, a cytochrome b₆-f complex, a plastocyanin, and a photosystem 1 (PS1) complex.

23. The in vitro cell-free system of claim 22, wherein the PS1 complex comprises the polypeptides PsaA-PsaS and the molecules chlorophyll 700, phylloquinone, Fe₄S₄, and carotenoid; and the cytochrome b₆-f complex comprises cytochrome b₆, cytochrome f, iron-sulfur protein, cytochrome b₆-f subunit IV, and the molecules heme b_{L}, heme b_{H}, heme c, and Fe₂S₂.

24. The in vitro cell-free system of claim 22, further comprising a photosystem 2 (PS2) complex and a plastoquinone.

25. The in vitro cell-free system of claim 16, wherein the ATP regeneration system comprises an ATP synthase, a photosystem 2 (PS2) complex, a plastoquinone, and a cytochrome b₆-f complex.

26. The in vitro cell-free system of claim 24, wherein the PS2 complex comprises the polypeptides Cp43, Cp47, PsbO, PsbP, PsbQ, PsbE, PsbF, manganese stabilizing protein, and the molecules chlorophyll 680, pheophytin, quinone, beta carotene, and heme b559.

27. The in vitro cell-free system of claim 16, wherein the enzymes comprise polypeptides obtained from a lysate or synthesized by in vitro translation.

28. The in vitro cell-free system of claim 16, wherein the energy source comprises light energy, glucose, ATP, or a combination thereof.

29. The in vitro cell-free system of claim 16, wherein the compound is a therapeutic compound, a precursor for a therapeutic compound or a dye.

30. The in vitro cell-free system of claim 29, wherein the compound is shikimic acid.

31. The in vitro cell-free system of claim 29, wherein the system comprises an enzyme selected from the group consisting of 3-deoxy-D-arabino-heptulosonate (DAHP) synthase, dehydroquinate synthase, dehydroquinate dehydratase, dehydroshikimate dehyrdogenase, and combinations thereof.

32. The in vitro cell-free system of claim 16, further comprising protease inhibitors, amino acids, a ribosome, an RNA encoding the amino acid sequence of an enzyme that catalyzes the formation of a compound, an RNA polymerase, a DNA encoding the nucleotide sequence of an enzyme that catalyzes the formation of a compound, or a combination thereof.

33. A method for synthesizing a compound, the method comprising:
providing the components comprising a source of carbon, a source of phosphate, water, enzymes that catalyze the formation of a compound, an energy source, adenosine diphosphate (ADP), and an adenosine triphosphate (ATP) regeneration system to form an in vitro cell-free reaction; and incubating the in vitro cell-free reaction to synthesize the compound.

34. The method of claim 33, wherein the ATP regeneration system is a photosynthetic ATP regeneration system.

35. The method of claim 33, wherein the ATP regeneration system is isolated from a plant, an alga, or a cyanobacterium.

36. The method of claim 35, wherein the ATP regeneration system comprises a thylakoid membrane.

37. The method of claim 35, wherein the ATP regeneration system comprises chloroplasts.

38. The method of claim 33, wherein the ATP regeneration system comprises an ATP synthase, a cytochrome b₆-f complex, a plastocyanin, and a photosystem 1 (PS1) complex.

39. The method of claim 38, wherein the PS1 complex comprises the polypeptides PsaA-PsaS and the molecules chlorophyll 700, phylloquinone, Fe₄S₄, and carotenoid; and the cytochrome b₆-f complex comprises cytochrome b₆, cytochrome f, iron-sulfur protein, cytochrome b₆-f subunit IV, and the molecules heme b_{L}, heme b_{H}, heme c, and Fe₂S₂.

40. The method of claim 38, further comprising a photosystem 2 (PS2) complex and a plastoquinone.

41. The method of claim 33, wherein the ATP regeneration system comprises an ATP synthase, a photosystem 2 (PS2) complex, a plastoquinone, and a cytochrome b₆-f complex.

42. The method of claim 40, wherein the PS2 complex comprises the polypeptides Cp43, Cp47, PsbO, PsbP, PsbQ, PsbE, PsbF, manganese stabilizing protein, and the molecules chlorophyll 680, pheophytin, quinone, beta carotene, and heme b559.

43. The method of claim 33, wherein the enzymes comprise polypeptides obtained from a lysate or synthesized by in vitro translation.

44. The method of claim 33, wherein the energy source comprises light energy, glucose, ATP, or a combination thereof.

45. The method of claim 33, wherein the compound is a therapeutic compound, a precursor for a therapeutic compound, or a dye.

46. The method of claim 45, wherein the compound is shikimic acid.

47. (canceled)

48. The method of claim 45, wherein the in vitro cell-free reaction comprises an enzyme selected from the group consisting of 3-deoxy-D-arabino-heptulosonate (DAHP) synthase, dehydroquinate synthase, dehydroquinate dehydratase, dehydroshikimate dehyrdogenase, and combinations thereof.

49. The method of claim 33, wherein the in vitro cell-free reaction further comprises providing a component selected from the group consisting of protease inhibitors, amino acids, a ribosome, an RNA encoding the amino acid sequence of an enzyme that catalyzes the formation of a compound, an RNA polymerase, a DNA encoding the nucleotide sequence of an enzyme that catalyzes the formation of a compound, and combinations thereof.

50. The method of claim 33, wherein the carbon source is a precursor for the compound.

51. The method of claim 33, wherein the components are added in more than one step.

52. The method of claim 33, further comprising a step for purifying the synthesized compound.

53-55. (canceled)

56. The method of claim 54, the method further comprising providing light energy to produce ATP from ADP using a photosynthetic ATP regeneration system.

57-65. (canceled)

66. The method of claim 46, wherein the in vitro cell-free reaction comprises an enzyme selected from the group consisting of 3-deoxy-D-arabino-heptulosonate (DAHP) synthase, dehydroquinate synthase, dehydroquinate dehydratase, dehydroshikimate dehydrogenase, and combinations thereof.

67. The method of claim 46, wherein the precursor for shikimic acid is phosphoenolpyruvate (PEP), erythrose 4-phosphate (E4P), 3-deoxy-D-arabino-heptulosonate (DAHP), dehydroquinic acid, dehydroshikimic acid, or a combination thereof.

68. (canceled)

69. (canceled)

70. The method of claim 46, further comprising a step for purifying shikimic acid.

71. (canceled)

72. (canceled)

73. The isolated composition of claim 15, wherein the ATP regeneration system comprises pyruvate kinase (PK).

74. The isolated composition of claim 73, wherein the ATP regeneration system further comprises phosphoglucose isomerase, phosphofructokinase (PKK-1), frustose bisphosphate aldolase, triosephosphate isomerase (TPI), glyceraldehyde phosphate dehydrogenase (GADPH), phosphoglycerate kinase (PGK), phosphoglycerate mutase (PGM), and enolase.

75. The isolated composition of claim 74, wherein the ATP regeneration system further comprises hexokinase (HK).

76. (canceled)

77. (canceled)

78. The in vitro cell-free system of claim 31, wherein the ATP regeneration system comprises pyruvate kinase (PK).

79. The in vitro cell-free system of claim 78, wherein the ATP regeneration system further comprises phosphoglucose isomerase, phosphofructokinase (PKK-1), frustose bisphosphate aldolase, triosephosphate isomerase (TPI), glyceraldehyde phosphate dehydrogenase (GADPH), phosphoglycerate kinase (PGK), phosphoglycerate mutase (PGM), and enolase.

80. The in vitro cell-free system of claim 79, wherein the ATP regeneration system further comprises hexokinase (HK).

81. The in vitro cell-free system of claim 78, wherein the energy source comprises glucose, glucose 6-phosphate, phosphoenolpyruvate, or a combination thereof.

82. (canceled)

83. The method of claim 48, wherein the ATP regeneration system comprises pyruvate kinase (PK).

84. The method of claim 82, wherein the ATP regeneration system further comprises phosphoglucose isomerase, phosphofructokinase (PKK-1), frustose bisphosphate aldolase, triosephosphate isomerase (TPI), glyceraldehyde phosphate dehydrogenase (GADPH), phosphoglycerate kinase (PGK), phosphoglycerate mutase (PGM), and enolase.

85. The method of claim 84, wherein the ATP regeneration system further comprises hexokinase (HK).

86. The method of claim 83, wherein the energy source comprises glucose, glucose 6-phosphate, phosphoenolpyruvate, or a combination thereof.
